(19) United States
(12) Patent Application Publication
(10) Pub. No.: US 2010/0151499 A1
(43) Pub. Date: Jun. 17, 2010

(54) PROTEOLYTIC RELEASE OF GLYCANS

(75) Inventors: Brian Edward Collins, Arlington, VA (US); Dorota A. Bulik, Winchester, MA (US); Carlos J. Bosques, Arlington, MA (US); Ian Christopher Parsons, Belmont, MS (US)

Correspondence Address: CHOATE, HALL & STEWART LLP, TWO INTERNATIONAL PLACE, BOSTON, MA, 02110, US

(73) Assignee: Momenta Pharmaceuticals, Inc., Cambridge, MA (US)

(21) Appl. No.: 12/595,946

(22) PCT Filed: Apr. 15, 2008
(86) PCT No.: PCT/US08/60333

§ 371 Date: Jan. 22, 2010

Related U.S. Application Data
(60) Provisional application No. 60/923,687, filed on Apr. 16, 2007.

(51) Int. Cl. G01N 33/567; C12P 21/00

(52) U.S. Cl. 435/7.21; 435/68.1

(57) ABSTRACT

The present disclosure provides strategies for analyzing protein-linked glycans, and particularly for analyzing glycans on cell surface glycoproteins.

### CROSS-REFERENCE TO RELATED APPLICATIONS

This application claims priority to U.S. provisional application, Ser. No. 60/923,687, filed Apr. 16, 2007, the entire contents of which are incorporated herein by reference.

### BACKGROUND

Cell surface carbohydrates found on most eukaryotic cells are thought to be involved in a variety of biological mechanisms, including cell differentiation and development, binding of viruses to the cell surface, tumor progression, and modulation of activity of cell surface receptors. Techniques for the detection and/or analysis of cell surface glycan structures are thus important tools.

### SUMMARY

The present disclosure provides systems for isolating, and/or analyzing cell surface glycan structures that are present on cell surface glycoproteins. For example, the present disclosure provides methods that release cell surface glycans without significantly rupturing the cell membrane, so that preparations of cell surface glycans can be prepared that are substantially free of glycans from non-cell-surface sources. In some embodiments, cell membranes remain substantially intact during release of glycans. In some particular embodiments, at least 80%, 85%, 90%, 95%, 96%, 97%, 98%, 99%, or more of the cell membranes remain intact (e.g., as monitored by trypan blue exclusion).

The present disclosure provides methods in which cells bearing cell surface glycoproteins are contacted with at least one protease that cleaves a cell surface glycoprotein so that at least one glycopeptide is released from the cell. According to the present disclosure, conditions can be selected to avoid rupturing cell membranes and/or to allow isolation of collections of cell surface glycopeptides that are substantially free of glycopeptides or glycoproteins from other than cell surface sources.

In some embodiments of the present disclosure, cells bearing at least one cell surface glycoprotein are contacted with at least one proteolytic enzyme that cleaves a cell surface glycoprotein so that at least one cell surface glycopeptide is released from the cell. In certain such embodiments, release of intracellular glycoproteins and/or glycopeptides is minimized so that contamination of liberated glycopeptides is reduced or eliminated. In certain embodiments, cells bearing at least one cell surface glycan attached to a cell surface glycoprotein are subjected to a labeling agent prior to contacting such cells with proteolytic enzymes, such that the cell surface glycan is labeled prior to liberation

In certain embodiments, treatment with a protease that does not rupture the cell membrane provides a substantially pure population of liberated cell surface glycopeptides, thus improving the overall characterization of cell surface glycans as compared with characterization achieved by standard methods.

In some embodiments, at least 50%, 60, 70, 80, 90% of cell surface glycoproteins are cleaved, such that approximately 50%, 60%, 70%, 80%, 90% of cell surface glycans are released from the cell.

In certain embodiments, cell surface glycopeptides are liberated by subjecting a cell to a plurality of proteases. Individual proteases within such a plurality of proteases may be administered simultaneously and/or sequentially. In some embodiments, simultaneous protease treatment results in greater (e.g., at least about 2%, 3%, 4%, 5%, 6%, 7%, 8%, 9%, 10%, 11%, 12%, 13%, 14%, 15% or more) release of glycans. Also, in some embodiments, treatment with a plurality of proteases results in higher levels of release than is observed with any of the individual proteases alone. In some embodiments, combinations of proteases result in at least 50%, 60%, 70%, 80%, 90%, 91%, 92%, 93%, 94%, 95%, 96%, 97%, 98%, 99% or more of cell surface glycans.

It will be clear to those of ordinary skill in the art that, among other things, methods described herein provide improvements over currently available methods for analyzing cell surface glycans including improved accuracy due to decreased (and in some embodiments substantial lack of) contamination with glycans from other sources, greater accuracy of relative quantitation of glycan structure. For example, by avoiding contamination with intracellular glycans, which can include, among other things, immature forms of glycoconjugates etc., the present disclosure allows a more accurate assessment of the true glycosylation pattern present on the surface of a cell.

The present disclosure also provides methods that allow assessment of glycan structure, particularly of cell surface glycans, with a reduced number and/or complexity of manipulation steps required to isolate and/or detect the glycans.

In some embodiments of the present disclosure, glycan preparations achieved by protease treatment of cells contain less than 25%, 20%, 15%, 10%, 5% or less intracellular glycans.

### BRIEF DESCRIPTION OF THE DRAWING

FIG. 1 shows liquid chromatography analysis of labeled glycans isolated from CHO cell surface glycoproteins. The top panel shows labeled glycans isolated by treatment with trypsin. The bottom panel shows labeled glycans isolated by treatment with proteinase K.

FIG. 2 shows loss of cell surface glycoproteins after treatment with varying concentrations of proteinase K.

FIG. 3 shows liquid chromatography analysis of cell surface glycoprotein N-linked glycans following treatment with glycosidases: Panel A shows untreated glycans; Panel B shows glycans treated with sialidase; Panel C shows glycans treated with both sialidase and hexosaminidase.

### DEFINITIONS

Approximately, About, Ca.: As used herein, the terms “approximately”, “about” or “ca.,” as applied to one or more values of interest, refer to a value that is similar to a stated reference value. In certain embodiments, the terms “approximately”, “about” or “ca.,” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

Biological sample: The term “biological sample”, as used herein, refers to any solid or fluid sample obtained from, excreted by or secreted by any living cell or organism, including, but not limited to, tissue culture, bioreactors, human or animal tissue, plants, fruits, vegetables, single-celled microorganisms (such as bacteria and yeasts) and multicellular organisms. For example, a biological sample can be a biological fluid obtained from, e.g., blood, plasma, serum, urine, bile, seminal fluid, cerebrospinal fluid, aqueous or vitreous humor, or any bodily secretion, a transudate, an exudate (e.g., fluid obtained from an abscess or any other site of infection or inflammation), or fluid obtained from a joint (e.g., a normal joint or a joint affected by disease such as a rheumatoid arthritis, osteoarthritis, gout or septic arthritis). A biological sample can also be, e.g., a sample obtained from any organ or tissue (including a biopsy or autopsy specimen), can comprise cells (whether primary cells or cultured cells), medium conditioned by any cell, tissue or organ, tissue culture.

Cell-surface glycoprotein: As used herein, the term “cell-surface glycoprotein” refers to a glycoprotein, at least a portion of which is present on the exterior surface of a cell. In some embodiments, a cell-surface glycoprotein is a protein that is positioned on the cell surface such that at least one of the glycan structures is present on the exterior surface of the cell.

Cell-surface glycan: A “cell-surface glycan” is a glycan that is present on the exterior surface of a cell. In many embodiments of the present disclosure, a cell-surface glycan is covalently linked to a polypeptide as part of a cell-surface glycoprotein. A cell-surface glycan can also be linked to a cell membrane lipid.

Glycan: As is known in the art and used herein “glycans” are sugars. Glycans can be monomers or polymers of sugar residues, but typically contain at least three sugars, and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

Glycan preparation: The term “glycan preparation” as used herein refers to a set of glycans obtained according to a particular production method. In some embodiments, glycan preparation refers to a set of glycans obtained from a glycoprotein preparation (see definition of glycoprotein preparation below).

Glycoconjugate: The term “glycoconjugate”, as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

Glycoform: The term “glycoform”, is used herein to refer to a particular form of a glycoconjugate. That is, when the same backbone moiety (e.g., polypeptide, lipid, etc.) that is part of a glycoconjugate has the potential to be linked to different glycans or sets of glycans, then each different version of the glycoconjugate (i.e., where the backbone is linked to a particular set of glycans) is referred to as a “glycoform”.

Glycolipid: The term “glycolipid” as used herein refers to a lipid that contains one or more covalently linked sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may be comprised of one or more branched chains. In certain embodiments, sugar moieties may include sulfate and/or phosphate groups. In certain embodiments, glycoproteins contain O-linked sugar moieties; in certain embodiments, glycoproteins contain N-linked sugar moieties.

Glycoprotein: As used herein, the term “glycoprotein” refers to a protein that contains a peptide backbone covalently linked to one or more sugar moieties (i.e., glycans). As is understood by those skilled in the art, the peptide backbone typically comprises a linear chain of amino acid residues. In certain embodiments, the peptide backbone spans the cell membrane, such that it comprises a transmembrane portion and an extracellular portion. In certain embodiments, a peptide backbone of a glycoprotein that spans the cell membrane comprises an intracellular portion, a transmembrane portion, and an extracellular portion. In certain embodiments, methods of the present disclosure comprise cleaving a cell surface glycoprotein with a protease to liberate the extracellular portion of the glycoprotein, or a portion thereof, wherein such exposure does not substantially rupture the cell membrane. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties. In certain embodiments, methods disclosed herein comprise a step of analyzing any or all of cell surface glycoproteins, liberated fragments (e.g., glycopeptides) of cell surface glycoproteins, cell surface glycans attached to cell surface glycoproteins, peptide backbones of cell surface glycoproteins, fragments of such glycoproteins, glycans and/or peptide backbones, and combinations thereof.

Glycoprotein preparation: A “glycoprotein preparation”, as that term is used herein, refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

Glycosidase: The term “glycosidase” as used herein refers to an agent that cleaves a covalent bond between sequential sugars in a glycan or between the sugar and the backbone moiety (e.g., between sugar and peptide backbone of glycoprotein). In some embodiments, a glycosidase is an enzyme. In certain embodiments, a glycosidase is a protein (e.g., a protein enzyme) comprising one or more polypeptide chains. In certain embodiments, a glycosidase is a chemical cleavage agent.

Glycosylation pattern: As used herein, the term “glycosylation pattern” refers to the set of glycan structures present on a particular sample. For example, a particular glycoconjugate (e.g., glycoprotein) or set of glycoconjugates (e.g., set of glycoproteins) will have a glycosylation pattern. In some embodiments, reference is made to the glycosylation pattern of cell surface glycans. A glycosylation pattern can be characterized by, for example, the identities of glycans, amounts (absolute or relative) of individual glycans or glycans of particular types, degree of occupancy of glycosylation sites, etc., or combinations of such parameters.

Isotopically labeled: As used herein, an “isotopically labeled” agent (which may also posses properties such as a fluorophore, a heterobifunctional ligand, or a chromophore) is a reagent which bears an atom or set of atoms which have been isotopically enriched by chemical or enzymatic methods. For example, an isotopically labeled agent may be isotopically enriched by direct replacement of an atom, either by chemical or enzymatic methods, with an isotopic label (e.g., a proton (¹H) with a deuterium (²H) atom or atoms; a proton (¹H) with a tritium (³H) atom or atoms; a ¹²C with a ¹³C atom or atoms; a ¹⁶O with a ¹⁸O atom or atoms; a ¹⁴N with a ¹⁵N atom or atoms; a ³¹P with a ³²P atom or atoms, and the like), or by chemical synthesis. Exemplary isotopic labels include, but are not limited to, ²H, ³H, ¹³C_{,} ¹⁸O, ¹⁵N, ¹⁸O, ³³S, ³⁴S, ³²P, ²⁹Si, and ³⁰Si. Furthermore, a glycan preparation may be “isotopically labeled” by reaction with an isotopically labeled agent (i.e., the reaction transfers an isotopic label to the glycan moiety).

N-glycan: The term “N-glycan,” as used herein, refers to a polymer of sugars that has been released from a glycoconjugate but was formerly linked to the glycoconjugate via a nitrogen linkage (see definition of N-linked glycan below).

N-linked glycans: N-linked glycans are glycans that are linked to a glycoconjugate via a nitrogen linkage. A diverse assortment of N-linked glycans exists, but is typically based on the common core pentasaccharide (Man)₃(GlcNAc)(GlcNAc).

O-glycan: The term “O-glycan,” as used herein, refers to a polymer of sugars that has been released from a glycoconjugate but was formerly linked to the glycoconjugate via an oxygen linkage (see definition of O-linked glycan below).

O-linked glycans: O-linked glycans are glycans that are linked to a glycoconjugate via an oxygen linkage. O-linked glycans are typically attached to glycoproteins via N-acetyl-D-galactosamine (GaINAc) or via N-acetyl-D-glucosamine (GlcNAc) to the hydroxyl group of L-serine (Ser) or L-threonine (Thr). Some O-linked glycans also have modifications such as acetylation and sulfation. In some instances O-linked glycans are attached to glycoproteins via fucose or mannose to the hydroxyl group of L-serine (Ser) or L-threonine (Thr).

Phosphorylation: As used herein, the term “phosphorylation” refers to the process of covalently adding one or more phosphate groups to a molecule (e.g., to a glycan).

Polypeptide: In general, the term “polypeptide” refers to a string of at least two amino acids linked to one another by peptide bonds. As used herein, the term is specifically used to refer to a portion of a larger polypeptide (i.e., a protein) that is released by proteolytic cleavage of the protein. Such a polypeptide is in fact a fragment of the parent protein.

Protease: The term “protease” as used herein refers to an agent that cleaves a peptide bond between sequential amino acids in a polypeptide chain. In some embodiments, a protease is an enzyme (i.e., a proteolytic enzyme). In certain embodiments, a protease is a protein (e.g., a protein enzyme) comprising one or more polypeptide chains. In certain embodiments, a protease is a chemical cleavage agent.

Protein: In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

Resin: As used herein, a “resin” is an organic polymer. The polymer may be naturally occurring or synthetic.

Sialic acid: The term “sialic acid,” as used herein, is a generic term for the N- or O-substituted derivatives of neuraminic acid, a nine-carbon monosaccharide. The amino group of neuraminic acid typically bears either an acetyl or a glycolyl group in a sialic acid. The hydroxyl substituents present on the sialic acid may be modified by acetylation, methylation, sulfation, and phosphorylation. The predominant sialic acid is N-acetylneuraminic acid (Neu5Ac). Sialic acids impart a negative charge to glycans, because the carboxyl group tends to dissociate a proton at physiological pH. Exemplary deprotonated sialic acids are as follows:

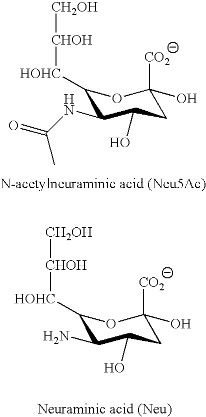

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena. To give but one particular example, when it is said that a treatment does not “substantially” rupture the cell membranes, it is meant to indicate that all or most of the cell membranes remain intact during and after the treatment, for example so that intracellular glycoproteins or glycopeptides are thus not released from the cells. In certain embodiments, the term “substantially”, as applied to unruptured cell membranes, refers to condition wherein 15%, 10%, 9%, 8%, 7%, 6%, 5%, 4%, 3%, 2%, 1%, or fewer of the cells subjected to a particular treatment exhibit measurable ruptured cell membranes. In certain embodiments, the term “substantially”, as applied to unruptured cell membranes, refers to condition wherein none of the cells subjected to a particular treatment exhibit measurable ruptured cell membranes.

### DETAILED DESCRIPTION OF CERTAIN EMBODIMENTS

The present disclosure provides systems for isolating and/or analyzing cell surface glycans by exposing cells to one or more proteases that cleave cell surface glycoproteins and release glycopeptides containing the glycans.

### Glycosylation of Proteins

Many proteins that are produced in a cell, including many presented on the cell surface, are glycoproteins comprising one or more glycans covalently attached to the polypeptide backbone. As will be appreciated by those of ordinary skill in the art, there can be a significant degree of heterogeneity in the glycan structures that are attached to glycoproteins.

Typically, N-linked oligosaccharide chains are added to a glycoprotein in the lumen of the endoplasmic reticulum (see Molecular Biology of the Cell, by Alberts et al., 1994, incorporated herein by reference). The oligosaccharide is added to the amino group on the side chain of an asparagine residue contained within the target consensus sequence of Asn-X-Ser/Thr, where X may be any amino acid except proline. The initial oligosaccharide chain is usually trimmed by specific glycosidase enzymes in the endoplasmic reticulum, resulting in a short, branched core oligosaccharide composed of two N-acetylglucosamine and three mannose residues. N-glycans can be subdivided into three distinct groups called “high mannose type”, “hybrid type”, and “complex type”, with a common pentasaccharide core (Man(alpha1,6)-(Man(alpha1,3))-Man(beta1,4)-GleNAc(beta1,4)- GlcNAc(betal,N)-Asn) occurring in all three groups.

After initial processing in the endoplasmic reticulum, the glycoprotein is then transported to the Golgi where further processing may take place. The trimmed N-linked oligosaccharide chain may be modified by the addition of several mannose residues, resulting in a “high-mannose glycan”.

Additionally or alternatively, one or more monosaccharides units of N-acetylglucosamine may be added to the core mannose subunits to form a “complex glycan”. Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

O-linked oligosaccharide chains are added to specific serine or threonine residues. The transfer of the first sugar residue, which in many instances is an N-acetylgalactosamine, typically begins in the endoplasmic reticulum and is completed in the Golgi apparatus. The residues of an O-linked oligosaccharide are added one at a time and the addition of each residue is catalyzed by a specific enzyme. In contrast to N-linked glycosylation, the consensus amino acid sequence for O-linked glycosylation is less well defined.

### Protease Treatment

The present disclosure provides strategies for isolating and/or analyzing glycans that are linked to proteins by cleaving the proteins so that glycopeptides are released. Methods described herein are particularly applicable to the isolation and/or characterization of cell surface glycans (e.g., glycans on cell surface glycoproteins).

In certain embodiments of the present disclosure, cell surface glycans are liberated from a cell surface by subjecting the cell to one or more proteases. Methods disclosed herein are particularly advantageous in that such methods permit release of glycan-containing glycopeptides, or portions thereof, from the cell surface, which glycopeptides and/or their component glycans and/or peptide backbones can be analyzed.

It is known in the art that glycan moieties present in glycoproteins commonly decrease access of proteases to the glycoprotein. Thus, prior to the present disclosure, it would typically be expected that subjecting cells to protease treatment would result in preferential release of non-glycosylated peptides as contrasted with glycopeptides. Thus, according to such traditional thinking, increased stringency of protease treatment (e.g., involving elevated levels of proteases) is expected to be required in order to release glycopeptides from cells. However, such increased stringency can result in rupture of cell membranes.

The present disclosure surprisingly demonstrates that protease treatment can be utilized to liberate glycopeptides from cells without significantly rupturing cell membranes. Non-limiting examples of applications of methods disclosed herein are described in the section below entitled “Applications”, as well as throughout the remainder of this specification. Those of ordinary skill in the art will appreciate the utility of methods described herein, and will recognize these and many other useful applications.

Proteases cleave amide bonds within a polypeptide chain. Several classes of proteases exist including both chemical and enzymatic agents. Proteolytic enzymes include, for example, serine proteases, threonine proteases, cysteine proteases, aspartic acid proteases, metalloproteases, and glutamic acid proteases. Non-limiting examples of specific proteolytic enzymes that can be used in accordance with the present disclosure include trypsin, chymotrypsin, elastase, subtilisin, proteinase K, pepsin, ficin, bromelin, plasmepsin, renin, chymosin, papain, a cathepsin (e.g. cathepsin K), a caspase (e.g. CASP3, CASP6, CASP7, CASP14), calpain 1, calpain 2, hermolysin, carboxypeptidase A or B, matrix metalloproteinase, a glutamic acid protease, and/or combinations thereof. Those of ordinary skill in the art will be aware of a number of other proteases that can be used in accordance with the present disclosure to release a glycoprotein from the surface of a cell.

Current methods of analyzing cellular glycoproteins, even when explicitly stated to be targeting cell surface glycans, are typically not particularly selective for cell surface glycans. For example, current methods typically employ one or more harsh detergents to extract membrane proteins, after which free sugars are dialyzed away before treatment with agents that remove glycan structures from proteins or polypeptides. Under such conditions, glycan preparations arc contaminated by intracellular glycans, e.g., from the endoplasmic reticulum and/or Golgi apparatus. Such intracellular glycans are typically immature, high-mannose glycans. Thus, their inclusion can skew the analysis of glycan structures associated with cell surface glycoproteins.

In certain embodiments of the present disclosure, glycans (in the form of glycopeptides) are liberated from a cell surface by subjecting the cell to one or more proteases under conditions that minimize disruption of the cell membrane. For example, in some embodiments, at least 80%, 85%, 90%, 95%, 96%, 97%, 98%, 99%, or more of the cell membranes remain intact (e.g., as monitored by trypan blue exclusion). Such methods are advantageous, among other things, because the can reduce or eliminate contamination from immature, high-mannose glycoproteins that are present inside the cell.

In some embodiments of the disclosure, glycans are liberated from a cell surface by subjecting the cell to one or more proteases for a limited period of time in order to avoid substantial lysis of the cell membrane. In certain embodiments, a cell is subjected to one or more proteases for a sufficiently limited time such that substantial lysis of the cell membrane does not occur.

For example, a cell may be subjected to one or more proteases for a period of time that is less than about 15, 14, 13, 12, 11, 10, 9, 8, 7, 6, 5, 4, 3, 2, or 1 minute. In certain embodiments, a cell is subjected to one or more proteases for a period of time that is more than 15 minutes so long as substantial lysis of the cell membrane does not occur. For example, a sufficiently low concentration of protease(s), a sufficiently low temperature and/or any of a variety of other factors or conditions may be employed such that the overall protease activity is decreased to a point where substantial lysis of the cell membrane does not occur. Those of ordinary skill in the art will be aware of and will be able to employ factors or conditions that ensure that substantial lysis of the cell membrane does not occur.

In certain embodiments of the present disclosure, protease cleavage is performed such that at least about 50%, 60%, 70%, 80%, 90%, 91%, 92%, 93%, 94%, 95%, 96%, 97%, 98%, 99% or more of cell surface glycans are released. To give but one specific example, the present disclosure demonstrated, for instance, that cleavage with trypsin for 15 min at 37 ° C. results in release of greater than 50% of the cell surface glycans.

In certain embodiments, cell surface glycans are liberated by subjecting a cell to one or more proteases (e.g., proteolytic enzymes) at a concentration of at least about 0.1 mg/mL. In certain embodiments, cell surface glycans are liberated by subjecting a cell to one or more proteases (e.g., proteolytic enzymes) at a concentration of less than about 2.0 mg/mL. In certain embodiments, cell surface glycans are liberated by subjecting a cell to one or more proteases (e.g., proteolytic enzymes) at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 5.0, 6.0, 7.0, 8.0, 9.0, 10.0 mg/mL or higher.

In certain embodiments, cell surface glycans are liberated by subjecting a cell to a plurality of proteases. For example, a cell may be subjected to 2, 3, 4, 5, 6, 7, 8, 9, 10, or more proteases to liberate cell surface glycans. Such a plurality of proteases may be administered to the cell simultaneously and/or sequentially. In certain embodiments, cell surface glycans are liberated by subjecting a cell to a plurality of proteases simultaneously, after which the liberated glycans (in the form of glycopeptides) are purified away from the cell.

In certain embodiments, cell surface glycans are liberated by subjecting a cell to a first protease (or plurality of first proteases) for a first period of time, after which the cell is subjected to a second protease (or plurality of second proteases) for a second period of time. Prior to treatment with the second protease, the first protease may optionally be removed and/or inactivated. By way of example, the first protease may be inactivated incubating the protease at a temperature for a time sufficient to inactivate it. Additionally or alternatively, the first protease may be inactivated by incubating it with an inhibitor that is specific to the protease (e.g. an antibody or other molecule that specifically binds the first protease and inhibits its catalytic activity). Other methods of inactivating the first protease will be known to those of ordinary skill in the art. In the case where the first protease is inactivated by incubating it with a specific inhibitor, it will be appreciated that the presence of the inhibitor should not substantially inhibit the activity of the second protease.

### Labeling Glycans

The present disclosure provides methods for isolating and/or analyzing cell surface glycans that are linked to glycoproteins and/or glycopeptides. In certain embodiments, methods of the present disclosure comprise a step of labeling such cell surface glycans to facilitate isolation and/or analysis.

According to the present disclosure, cell surface glycans may be labeled by any of a variety of methods and/or at any of a variety of times. In certain embodiments, cell surface glycans that are part of a glycoprotein produced by a cell are labeled by the cell during production of the glycoprotein. For example, a cell may be provided with a labeled sugar or sugar derivative, which labeled sugar or sugar derivative is used in the production of the glycoprotein, such that the produced glycoprotein comprises the label that was initially present on the sugar or sugar derivative. In certain embodiments, a plurality of sugars or sugar derivatives, each comprising a distinct label, is provided to a cell, such that one or more glycoproteins is produced that comprise one or more of the distinct labels. Such embodiments are useful for, among other applications, differentially labeling different glycoproteins that comprise different numbers and/or types of glycans. As described in more detail below, exemplary labels that can be used in such embodiments include radiolablels and/or fluorescent labels, although those of ordinary skill in the art will recognize that methods disclosed herein are not limited to use of such labels.

In certain embodiments, glycans are labeled on the cell surface after being incorporated into a glycoprotein. In some embodiments of the present disclosure, glycans are analyzed after being released from the cell surface, for example by protease treatment that releases glycopeptides, by glycanase treatment (e.g., exposure to an agent such as PNGase F, PNGase A, End-H, O-glycanase, hydrazine, sodium borohydride, endoglycosidases, trifluoromethasenulfonic acid (TFMS), and/or beta-elimination, etc.) that releases glycans, by protease treatment followed by glycanase treatment, or by combinations thereof In some embodiments, the protease treatment is performed such that it does not substantially rupture cell membranes. Regardless of the mode of treatment, glycans may be labeled at any stage. For example, glycans may be labeled prior to any treatment, or after any particular treatment (e.g., after protease treatment and/or after glycanase treatment).

Exemplary labeling agents that are useful for labeling glycoproteins, glycopeptides and/or glycans are described more fully below. However, as will be recognized by those of ordinary skill in the art, methods of the present disclosure are not limited to such exemplary labeling agents.

A “labeling agent”, as used herein, refers to a reagent which is capable of transferring a functional group (i.e., a label) to a glycan or a glycan preparation (e.g., a cell-surface glycan or cell-surface glycan preparation). In certain embodiment, upon its transfer, the label enhances the cell-surface glycan or cell-surface glycan preparation's detection by analytical or preparative methods, such as chromatography, nuclear magnetic resonance spectroscopy, and/or mass spectrometry. Thus, in certain embodiments, the “label” may be an isotope, a charged moiety, a UV-active or fluorescent functional group, or a combination thereof. In certain embodiments, the labeling agent is a UV-active or fluorescent tag that optionally includes an isotopic label (e.g., in the form of one or more atoms selected from ²H, ³H, ¹³C, ¹⁸O, ¹⁵N, ¹⁸O, ³³S, ³⁴S, ³²P, ²⁹Si, ³⁰S, etc.). The ability to detect the labeling agent (and any glycan to which it has been attached) by spectroscopic methods provides a useful handle for tracking a cell-surface glycan species within a cell-surface glycan preparation (e.g., during a preliminary chromatographic separation step).

In certain embodiments, a “labeling agent” as used herein refers to a reagent that is used by the cell in the production of a glycan, for example a glycan that is a component of a glycoprotein, which produced glycan can then be isolated and/or analyzed according to one or more methods of the present disclosure. In certain embodiments, a labeling agent comprises a sugar derivative such as, without limitation, fucose, mannose, N-acetyl glucosamine, N-mannosamine, N-acetyl neuraminic acid, N-glycolyl neuraminic acid and/or galactose derivatives that is used by the cell to introduce a reactive moiety. In some embodiments, methods of the present disclosure include introducing a precursor sugar (e.g., glucose, N-mannosamine, N-acetylglucosamine, neuraminic acid, galactose, mannose, fucose, hexose, arabinose, ribose, xylose, etc. and/or modified sugars such as 2′-fluororibose, 2′-deoxyribose, phosphomannose, 6′sulfo n-acetylglucosamine, etc.) that is converted by the cell into the final sugar and transferred onto the oligosaccharide. In certain of such embodiments, an N-mannosamine derivative such as, but not limited to, N-azido mannosamine, N-levulonyl mannosamine, and/or N-arylazido mannosamine, is converted by the cell into a sialic acid derivative such as, without limitation, an azido, a levulonyl, a ketone, an arylazido, and/or an azirine substitutions. In certain embodiments, substitutions may occur; exemplary, nonlimiting examples include N-substitutions or O-substitutions to the 1, 2, 3, 4, 5, 6, 7, 8, or 9 position of the sugar. For example, sugars may be derivatized in their 1,-9 positions. Such sugars may be incorporated into either N- or O-linked glycans, or both. Such reactive species can be specifically labeled using an orthogonal reactive moiety such as, without limitation, an akyne or phosphine that carries a label, such as without limitation, one or more of the labels described herein. Exemplary labels include, but are not limited to, fluorescent tags, enzyme tags (peroxidase, phosphatase, for example), peptide tags (His, FLAG, GST, Fc for example), radiochemical tags ¹⁴C, ¹²⁵I, ³²P, ³³P, ³H, ³⁵S for example), or other tag (biotin, PEG, acyl tags, NP, or streptavidin for example), or other tags known to those skilled in the art.

In some embodiments, glycans may be labeled in accordance with one or more methods of the present disclosure by introducing a sugar or precursor sugar that contains a radiolabel. Exemplary radiolabels include ¹⁴C, ¹²⁵I, ³²P, ³³P, ³H, and ³⁵S, although methods of the present disclosure are not limited to such exemplary radiolabels and those of ordinary skill in the art will be aware of other useful radiolabels that may be used in accordance with one or more methods disclosed herein. In some embodiments, glycans may be labeled in accordance with one or more methods of the present disclosure by incubating a cell with a labeled sugar or precursor sugar and allowing the cell to take up and incorporate the radiolabeled sugar or sugar precursor into the glycoprotein.

Glycans may be modified using chemical or enzymatic methods on the intact glycoprotein prior to release from the cell. In some embodiments, such treatment may involve treating the glycan with an enzymatic treatment to allow for incorporation of a label. As but one non-limiting example, an enzyme such as galactose oxidase may be used to introduce a radiochemical label. In some embodiments, an enzyme such as a glycosyltransferase may be used to transfer a label onto a sugar. Such sugars may be radiolabeled or tagged with a functional group as previously described. In certain embodiments, glycans may be labeled through the use of chemical treatment including, without limitation, mild sodium metaperiodate and/or NaBH₄ (or NaB³H₄) to introduce a radiolabel, chemical reactivity or labeled moiety.

In certain embodiments, the first labeling agent is isotopically labeled, and upon reaction with the first cell-surface glycan preparation provides a first isotopically-labeled cell-surface glycan preparation. In certain embodiments, the second labeling agent is isotopically labeled, and upon reaction with the second cell-surface glycan preparation provides a second isotopically-labeled cell-surface glycan preparation.

In certain embodiments, the first labeling agent is isotopically labeled and the second labeling agent is not isotopically labeled (i.e., it includes natural abundance atoms such as ¹II, ¹²C, ¹⁴N, etc.). In certain embodiments, both the first labeling agent and the second labeling agent are isotopically labeled.

In certain embodiments, the isotopic label is an atom selected from the group consisting of ²H, ³H, ¹³C, ¹⁵N, ¹⁸O, ³³S, ³⁴S, ³²P, ²⁹Si, and ³⁰Si. In certain embodiments, the isotopic label is ²H. In certain embodiments, the isotopic label is ³H. In certain embodiments, the isotopic label is ¹³C. In certain embodiments, the isotopic label is ¹⁵N. In certain embodiments, the isotopic label is ¹⁸O. In certain embodiments, the isotopic label is ³²P.

In one set of embodiments, the first labeling agent includes one isotopic label. In other embodiments, the first labeling agent may include more than one isotopic label. For example, it may include two different isotopic labels (e.g., ²H and ¹³C). Alternatively, the first labeling agent may include two or more copies of the same isotopic label (e.g., ²H).

In one set of embodiments, the second labeling agent is not isotopically labeled. In other embodiments, the second labeling agent may include one isotopic label. The second labeling agent may also include more than one isotopic label. For example, it may include two different isotopic labels (e.g., ²H and ¹³C). Alternatively, the second labeling agent may include two or more copies of the same isotopic label (e.g., ²H).

In certain embodiments, the first labeling agent and second labeling agent have the same chemical structure but include different isotopic labels. For example, the first labeling agent may be a deuterated (²H) or tritiated (³H) analog of the second labeling agent. In certain embodiments, the first labeling agent is a ¹³C analog of the second labeling agent. In certain embodiments, the first labeling agent is an ¹⁵N analog of the second labeling agent. In certain embodiments, the first labeling agent is an ¹⁸O analog of the second labeling agent. In certain embodiments, the first labeling agent is a ³²P analog of the second labeling agent.

In certain embodiments, the first and second labeling agent are fluorescent tags (i.e., each includes a fluorescent functional group). As mentioned above, fluorescent functional groups enable inter alia a higher sensitivity of detection of the glycan during chromatographic separation. Fluorescent tags for this purpose are described in the art; see, for example, Anumula, Anal. Biochem. (2006) 350:1-23; Lamari et al., J. Chromatogr. B (2003) 793:15-36; Bigge et al., Anal. Biochem. (1995) 230:229-238, and references provided therein.

Exemplary fluorescent tags include, but are not limited to, 2-aminobenzoic acid (2AA); 3-aminobenzoic acid (3AA); 4-aminobenzoic acid (4AA); anthranilic acid (AA); 2-aminopyridine (2AP); 2-aminobenzamide (2AB); 3-aminobenzamide (3AB); 4-aminobenzamide (4AB); 2-aminobenzoic ethyl ester (2ABEE); 3-aminobenzoic ethyl ester (3ABEE); 4-aminobenzoic ethyl ester (4ABEE); 2-aminobenzonitrile (2ABN); 3-aminobenzonitrile (3ABN); 4-aminobenzonitrile (4ABN); 3-(acetylamino)-6-aminoacridin (AA-AC); 2-aminoacridone (AMAC); methylanthranilate (MA); 6-aminoquinoline (6AQ); 8-aminonaphthalene-1,3,6-trisulfonic acid (ANTS); 2-aminonaphthalene-1,3,6-trisulfonate (ANT); 8-aminopyrene-1,3,6-trisulfonic acid (APTS); 7-aminomethyl-coumarin (AMC); 2-amino(6-amido-biotinyl)pyridine (BAP); 9-fluorenylmethoxy-carbonyl-hydrazide (FMOC-hydrazide); 3,5-dimethylanthranilic acid, and 2-amino-4,5-dimethoxy-benzoic acid.

In certain embodiments, the first labeling agent and second labeling agent are the same fluorescent tag but include different isotopes of one or more atom within the tag. For example, the first labeling agent is an isotopically enriched analog of the second labeling agent.

The present disclosure contemplates use of any and all known optionally isotopically labeled “labeling agents,” as provided above and herein.

### Labeled Glycan

According to the present disclosure, any of the aforementioned labeling agents may be used to label a cell-surface glycan in a glycan preparation such as, for example, a cell-surface glycoprotein preparation.

For example, a labeling agent that includes any of the above fluorescent tags may be used to label a cell-surface glycan via reaction of the amine function group of the labeling agent with the glycan's reducing (—CHO) end by reductive amination. One of ordinary skill in the art will appreciate that a wide variety of reaction conditions may be employed to promote a reductive amination reaction, therefore, a wide variety of reaction conditions are envisioned; see generally, March's Advanced Organic Chemistry: Reactions, Mechanisms, and Structure, M. B. Smith and J. March, 5^{th} Edition, John Wiley & Sons, 2001, and Comprehensive Organic Transformations, R. C. Larock, 2^{nd} Edition, John Wiley & Sons, 1999; and see specifically, Abdel-Magid et al., J. Org. Chem., (1996) 61:3849-3862 (e.g., use of sodium triacetoxyborohydride; NaBH₄/MeOH); McLaughlin et al., Org. Lett., (2006) 8:3307-3310 (e.g., an effective reductive alkylation of electron-deficient o-chloroarylamines); Mizuta et al., J. Org. Chem. (2005) 70:2195-2199 (e.g., methodology for the reductive alkylation of secondary amines with aldehydes and Et₃SiH using an iridium catalyst; use of an environmentally friendly reducing reagent polymethylhydrosiloxane (PMHS)); Menche et al., Org. Lett. (2006) 8:741-744 (e.g., direct reductive amination using selective imine activation with thiourea followed by transfer hydrogenation); Kumpaty et al, Synthesis (2003) 2206-2210 (e.g., a reductive mono-N-alkylation of primary amines with carbonyl compounds in the presence of Ti(i-PrO)₄ and NaBH₄); Miriyala et al., Tetrahedron (2004) 60:1463-1471 (e.g., reductive alkylation of ammonia with aldehydes); Sato et al., Tetrahedron (2004) 60:7899-7906 (e.g., one-pot reductive amination of aldehydes and ketones with amines using α-picolinc-boranc as a reducing agent in the presence of small amounts of AcOH, MeOH, and/or H₂O, and in neat conditions); and Bae et al., Chem. Commun. (2000) 1857-1858 (e.g., reduction of nitrobenzenes followed by reductive amination with decaborane (B₁₀H₁₄) in the presence of 10% Pd/C); the entirety of each of which is hereby incorporated herein by reference.

In certain embodiments, the reductive amination reaction is carried out in a suitable medium. A suitable medium is a solvent or a solvent mixture that, in combination with the combined reacting partners and reagents, facilitates the progress of the reaction therebetween. A suitable medium may solubilize one or more of the reaction components, or, alternatively, the suitable medium may facilitate the suspension of one or more of the reaction components; see, generally, March (2001). Solvents which may be used to facilitate the reductive amination reaction include, but are not limited to, ethers, halogenated hydrocarbons, aromatic solvents, esters, or mixtures thereof. In certain embodiments, the solvent is diethyl ether, dioxane, tetrahydrofuran (THF), dichloromethane (DCM), dichloroethane (DCE), chloroform, toluene, benzene, ethyl acetate, isopropyl acetate, or a mixture thereof.

In certain embodiments, the solvent medium further comprises one or more hydrogen-donating additives, such as an organic alcohol and/or an acid. Exemplary organic alcohols include, but are not limited to, methanol, ethanol, isopropanol, or t-butanol. Exemplary hydrogen donating acids include, but are not limited to, hydrogen chloride, hydrogen bromide, hydrogen iodide, hydrogen fluoride, formic acid, toluenesulfonic acid (TsOH), trifluoroacetic acid (TFA), and acetic acid (AcOH). In certain embodiments, the pH of the reaction solution is acidic. In certain embodiments, the pH of the reaction solution is between about 4 to about 6.

In certain embodiments, the solvent medium is buffered (e.g., contains a buffer such as NaOAc—AcOH). In certain embodiments, the pH of the buffered reaction solution is acidic. In certain embodiments, the pH of the buffered reaction solution is between about 4 to about 6.

In certain embodiments, the reductive amination reaction also comprises a catalyst, such as a Lewis acid catalyst or a transition metal catalyst. Exemplary Lewis acid catalysts include, but are not limited to, Ti(i-PrO)₄, AlCl₃, BCl₃, BBr₃, and FeCl₃. Exemplary transition metal catalysts include, but are not limited to, nickel, zinc, ruthenium, rhodium, palladium, platinum, gold, and mercury. In certain embodiments, the transition metal catalyst is a palladium catalyst. In certain embodiments, the palladium catalyst is palladium-on-carbon. In certain embodiments, the reductive amination reaction is conducted under hydrogen gas or deuterium gas pressure (e.g., between about 5 to about 80 psi).

In certain embodiments, the reducing agent used in the reductive amination is also isotopically labeled. In certain embodiments, the reducing agent is D₂-Pd/C, D₂-Raney Nickel, NaBD₄, B₁₀D₁₄, BD₃-pyridine, α-picoline-BD₂; NaBD₃CN, or NaBD(OCOCH₃)₃.

In certain embodiments, an isotopically labeled cell-surface glycan preparation may be prepared by reductive amination of an isotopically labeled fluorescent tag with a cell-surface glycan preparation using a reducing agent. According to such an embodiment, the resulting isotopically labeled cell-surface glycan preparation bears at least one isotopic label from the fluorescent tag.

In certain embodiments, an isotopically labeled cell-surface glycan preparation may be prepared by reductive amination of a fluorescent tag with a cell-surface glycan preparation using an isotopically labeled reducing agent. According to such an embodiment, the resulting isotopically labeled cell-surface glycan preparation bears at least one isotopic label from the reducing agent.

In certain embodiments, an isotopically labeled cell-surface glycan preparation may be prepared by reductive amination of an isotopically labeled fluorescent tag with a cell-surface glycan preparation using an isotopically labeled reducing agent. According to such an embodiment, the resulting isotopically labeled cell-surface glycan preparation bears at least one isotopic label from the fluorescent tag and at least one isotopic label from the reducing agent.

Labeling of a cell-surface glycan is not limited to derivatization of the reducing end by reductive amination. The present disclosure also provides suitable labeling agents for tagging other functional groups present on the glycan. For example, as depicted in Scheme 2, 1,2-diamino functionalized labeling agents, such as 1,2-diamino-4,5-methylenedioxy-benzene (DMB) and ortho-phenylenediamine (OPD), are suitable for tagging via reaction with the alpha-keto acid functional group of sialic acids.

The labeling reactions described above are just two of many ways to isotopically label a cell-surface glycan or a cell-surface glycan preparation, and, as presented herein, are understood to be non-limiting examples.

### Glycan Analysis

In many embodiments of the present disclosure, glycans present on glycopeptides released as described herein are subjected to one or more additional isolation, purification, and/or analysis steps. A wide variety of methodologies for such isolation, purification, and/or analysis are available in the art; one of ordinary skill would appreciate their applicability, as well as the applicability of other technologies, to applications as described herein. The present discussion is not intended in any way to represent an exhaustive list of possibilities, but rather to provide a representative or suggestive summary of some options.

In certain embodiments of the disclosure, cell surface glycans liberated from cells according to the present disclosure are then released from the liberated glycopeptides according to any of a variety of techniques. Any of a variety of glycosidic and other enzymes that cleave glycan structures from glycopeptides may be used in accordance with the present disclosure. Several examples of such enzymes are reviewed in R. A. O'Neill, Enzymatic release of oligosaccharides from glycoproteins for chromatographic and electrophoretic analysis, J. Chromatogr. A 720, 201-215. 1996; and S. Prime, et al., Oligosaccharide sequencing based on exo- and endo-glycosidase digestion and liquid chromatographic analysis of the products, J. Chromatogr. A 720, 263-274, 1996, each of which is incorporated herein by reference in its entirety. In certain embodiments, the enzyme PNGase F (Peptide N-Glycosidase F) is used to remove glycans from a glycopeptide or glycoprotein. PNGase F is an amidase that cleaves the amide bond between the innermost GlcNAc and asparagine residues of high mannose, hybrid, and complex oligosaccharides from N-linked glycoproteins. Additionally or alternatively, in certain embodiments, the enzymes PNGase A, O-glycanase and/or Endo-H are used to remove glycans.

To improve the accessibility of the glycosylation site to a cleavage enzyme, it may be desirable to include a denaturation step. Typically, this is accomplished by using detergents (e.g., SDS) and/or disulfide-reducing agents (e.g., beta-mercaptoethanol), although methods of denaturing a glycoprotein for use in accordance with the present disclosure are not limited to the use of such agents. For example, exposure to high temperature can be sufficient to denature a glycoprotein or glycopeptide such that a suitable enzyme for cleaving glycan structures is able to access the cleavage site. In certain embodiments, a glycoprotein or glycopeptide is denatured by incubation at a temperature of 80, 81, 82, 83, 84, 85, 86, 87, 88, 89, 90, 91, 92, 93, 94, 95, 96, 97, 98, 99, 100 degrees Celsius, or higher for a period of time sufficient to denature the glycoprotein or glycopeptide.

In certain embodiments, a combination of detergents, disulfide-reducing agents, high temperature, and/or other agents or reaction conditions is employed to denature a glycoprotein or glycopeptide. Those of ordinary skill in the art will be aware of suitable conditions, incubation times, etc. that will be sufficient to denature a glycoprotein or glycopeptide. It is noted that oligosaccharides located at conserved Fc sites in immunoglobulin G (IgG) are easily cleaved by PNGase F. Thus, a denaturation step is typically not required for IgG molecules when this enzyme is used. PNGase F is also capable of removing oligosaccharides in dilute ammonium hydroxide solution, is stable in 2.5M urea at 37 C for 24 h, and still possesses 40% of its activity in 5 M urea. Thus, PNGase F has the advantage that it is capable of cleaving glycans from glycoproteins or glycopeptides under certain denaturation conditions.

Other suitable enzymes that can be used to cleave glycan structures from glycopeptides in accordance with the present disclosure include, but are not limited to, PNGase A, O-glycanase and Endo-H. Those of ordinary skill in the art will be aware of other suitable enzymes for cleavage of glycans from glycopeptides. In certain embodiments, a plurality of enzymes is used to cleave glycan structures from a glycopeptide. In certain embodiments, such a plurality of cleavage enzymes is administered simultaneously. In certain embodiments, such a plurality of cleavage enzymes is administered sequentially.

In certain embodiments, one or more glycan structures are cleaved from released glycopeptides through the use of an agent other than an enzyme. In certain embodiments, a chemical agent or plurality of chemical agents can be used to cleave glycan structures from glycoproteins or glycopeptides. For example, use of the chemical hydrazine has been successfully employed to cleave glycan structures. As another non-limiting example, it has been suggested that a mixture of ammonia-ammonium carbonate can be used for alkaline release of both the N- and O-linked oligosaccharides in their native form (see Y. Huang, et al., Microscale nonreductive release of O-linked glycans for subsequent analysis through MALDI mass spectrometry and capillary electrophoresis, Anal. Chem. 73, 6063-60, 2001, incorporated herein by reference in its entirety). Those of ordinary skill in the art will be aware of other suitable chemical agents that can be used in accordance with the present disclosure. In some cases, use of a chemical agent to cleave glycan structures from a glycopeptide results in protein degradation as well as cleavage. However, after cleavage, the glycan structure is often purified away from the protein component of the glycopeptide before analysis and/or characterization. In such situations, degradation of the protein component after treatment with a chemical agent is not detrimental to the practice of the present disclosure. In some cases, degradation of the protein component may even aid in the process of purifying the cleaved glycan structure(s).

In certain embodiments, cell surface glycans liberated from cells according to the present disclosure are characterized one or more available methods (to give but a few examples, see Anumula, Anal. Biochem, 350(1):1-23, 2006; Klein et al. Anal. Biochem.,179:162-66, 1989; and Townsend, R. R., Carbohydrate Analysis: High Performance Liquid Chromatography and Capillary Electrophoresis, ed. Z. El Rassi, pp. 181-209, 1995, each of which in incorporated herein by reference in its entirety). For example, in some embodiments, such glycans are characterized by methods such as chromatographic methods, mass spectroscopic methods, electrophoretic methods, nuclear magnetic resonance (NMR) methods, and combinations thereof. For example, in some embodiments, glycans are characterized by one or more of NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof.

In some embodiments, N-glycan structure and composition can be analyzed by chromatographic methods, including but not limited to, liquid chromatography (LC), high performance liquid chromatography (HPLC), ultra performance liquid chromatography (UPLC), thin layer chromatography (TLC), amide column chromatography, and combinations thereof.

In some embodiments, N-glycan structure and composition can be analyzed by mass spectrometry (MS) and related methods, including but not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof.

In some embodiments, N-glycan structure and composition can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof.

In some embodiments, N-glycan structure and composition can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

Those of ordinary skill in the art will be aware of other methods that can be used to characterize glycosylation patterns of liberated cell surface glycoproteins.

In certain embodiments, cell surface glycoprotein and/or glycan structures are labeled prior to liberation and/or characterization. As is known to those of ordinary skill in the art, such labeling may increase signal and/or reduce background noise during characterization. Any of a variety of labels can be used in accordance with the present disclosure, including but not limited to, fluorescent labels, radiolabels and/or chemiluminescent labels. In certain embodiments, glycan structures are labeled with fluorescent 2-aminobenzamide (“2-AB”). Those of ordinary skill in the art will be aware of other suitable labels that can be used in accordance with the present disclosure.

In certain embodiments of the disclosure, glycan structures are labeled prior to analysis by mass spectrometry. In some such embodiments, different glycan preparations are labeled with labels of different mass. Such approaches can facilitate relative quantitation of glycan structures. For example, Example 2 describes determination of relative amounts of different glycan species provided in different cell-surface glycan preparations, e.g., protease digests of two different samples.

### Applications

It will be appreciated that methods described herein may be used in a variety of applications. In general, any application that requires the structural characterization and/or isolation of cell surface glycans linked to proteins, or indeed any application that would benefit from release of glycopeptides from an environment, may benefit from the present methods.

The present disclosure is particularly useful to provide a pool of peptide-linked glycans separate from the source in which the glycans were generated and/or with which the glycans were previously associated.

Methods of the present disclosure can be applied to cell surface glycans obtained from a wide variety of sources including, but not limited to, therapeutic formulations and biological samples containing cells. Such a biological sample may undergo one or more analysis and/or purification steps prior to or after being analyzed according to the present disclosure. To give but a few examples, in some embodiments, a biological sample is treated with one or more proteases and/or glycosidase (e.g., so that glycans are released); in some embodiments, glycans in a biological sample are labeled with one or more detectable markers or other agents that may facilitate analysis by, for example, mass spectrometry or NMR. Any of a variety of separation and/or isolation steps may be applied to a biological sample in accordance with the present disclosure.

Methods of the present disclosure can be utilized to analyze free glycans or glycans in the context of a glycoconjugate (e.g., a glycopeptide).

Methods of the present disclosure may be used in one or more stages of process development for the production of a therapeutic or other commercially relevant glycoprotein of interest. Non-limiting examples of such process development stages that can employ methods of the present disclosure include cell selection, clonal selection, media optimization, culture conditions, process conditions, and/or purification procedure. Those of ordinary skill in the art will be aware of other process development stages.

The present disclosure can also be utilized to monitor the extent and/or type of cell surface glycosylation occurring in a particular cell culture, thereby allowing adjustment or possibly termination of the culture in order, for example, to achieve a particular desired cell surface glycosylation pattern or to avoid development of a particular undesired cell surface glycosylation pattern.

The present disclosure can also be utilized to assess cell surface glycosylation characteristics of cells or cell lines that are being considered for production of a particular desired glycoprotein (for example, even before the cells or cell lines have been engineered to produce the glycoprotein, or to produce the glycoprotein at a commercially relevant level).

In some embodiments of the disclosure, a desired glycosylation pattern for a particular target glycoprotein (e.g., a cell surface glycoprotein) is known, and the technology described herein allows monitoring of culture samples to assess progress of the production along a route known to produce the desired glycosylation pattern. For example, where the target glycoprotein is a therapeutic glycoprotein, for example having undergone regulatory review in one or more countries, it will often be desirable to monitor cultures to assess the likelihood that they will generate a product with a glycosylation pattern as close to the established glycosylation pattern of the pharmaceutical product as possible, whether or not it is being produced by exactly the same route. As used herein, “close” refers to a glycosylation pattern having at least about a 75%, 80%, 85%, 90%, 95%, 98%, or 99% correlation to the established glycosylation pattern of the pharmaceutical product. In such embodiments, samples of the production culture are typically taken at multiple time points and are compared with an established standard or with a control culture in order to assess relative glycosylation.

In some embodiments, methods in accordance with the disclosure may be used to monitor the cell surface glycosylation pattern during culture of cells that produce a glycoprotein. For example, production of a glycoprotein (e.g., commercial production) may involve steps of (1) culturing cells that produce the glycoprotein, (2) obtaining samples at regular or irregular intervals throughout the process of culturing the cells, and (3) analyzing the cell surface glycosylation pattern on obtained samples. In some embodiments, such methods may further comprise a step of comparing the cell surface glycosylation patterns of different samples to one another. In some embodiments, such methods may further comprise a step of comparing the cell surface glycosylation patterns for one or more obtained samples to the glycosylation pattern of a reference sample.

In some embodiments of the present disclosure, a desired cell surface glycosylation pattern will be more extensive. For example, in some embodiments, a desired cell surface glycosylation pattern shows high (e.g., greater than about 60%, about 65%, about 70%, about 75%, about 80%, about 85%, about 90%, about 95%, about 98%, about 99%, or more) occupancy of cell surface glycosylation sites; in some embodiments, a desired cell surface glycosylation pattern shows, a high degree of branching (e.g., greater than about 60%, about 65%, about 70%, about 75%, about 80%, about 85%, about 90%, about 95%, about 98%, about 99% or more have tri or tetra-antennary structures).

In some embodiments of the present disclosure, a desired cell surface glycosylation pattern will be less extensive. For example, in some embodiments, a desired cell surface glycosylation pattern shows low (e.g., less than about 50%, about 45%, about 40%, about 35%, about 30%, about 25%, about 20%, about 15%, about 15%, about 5%, about 1%, or less) occupancy of cell surface glycosylation sites; and/or a low degree of branching (e.g., less than about 20%, about 15%, about 10%, about 5%, about 1% or less have tri or tetra-antennary structures).

In some embodiments, a desired cell surface glycosylation pattern will be more extensive in some aspects and less extensive in others. For example, it may be desirable to employ a cell line that tends to produce cell surface glycoproteins with long, unbranched oligosaccharide chains. Alternatively, it may be desirable employ a cell line that tends to produce cell surface glycoproteins with short, highly branched oligosaccharide chains.

In some embodiments, a desired glycosylation pattern will be enriched for a particular type of glycan structure. For example, in some embodiments, a desired glycosylation pattern will have low levels (e.g., less than about 20%, about 15%, about 10%, about 5%, about 1%, or less) of high mannose or hybrid structures, high levels (e.g., greater than about 60%, about 65%, about 70%, about 75%, about 80%, about 85%, about 90%, about 95%, about 98%, about 99%, or more) of high mannose structures, -high levels (e.g., greater than about 60%, about 65%, about 70%, about 75%, about 80%, about 85%, about 90%, about 95%, about 98%, about 99%, or more; for example at least one per glycoprotein) phosphorylated high mannose, or low levels (e.g., less than about 20%, about 15%, about 10%, about 5%, about 1%, or less) of phosphorylated high mannose.

In some embodiments, a desired glycosylation pattern will include at least about one sialic acid. In some embodiments, a desired glycosylation pattern will include a high level (e.g., greater than about 60%, about 65%, about 70%, about 75%, about 80%, about 85%, about 90%, about 95%, about 98%, about 99%, or more) level of termini that are sialylated. In some embodiments, a desired glycosylation pattern that includes sialyation will show at least about 85%, about 90%, about 95%, about 98%, about 99%, or more N-acetylneuraminic acid and/or less than about 20%, about 15%, about 10%, about 5%, about 1%, or less N-glycolylncuraminic acid.

In some embodiments, a desired glycosylation pattern shows specificity of branch elongation (e.g., greater than about 60%, about 65%, about 70%, about 75%, about 80%, about 85%, about 90%, about 95%, about 98%, about 99%, or more of extension is on α1,6 mannose branches; or greater than about 60%, about 65%, about 70%, about 75%, about 80%, about 85%, about 90%, about 95%, about 98%, about 99%, or more of extension is on α1,3 mannose branches).

In some embodiments, a desired glycosylation pattern will include a low level (e.g., less than about 20%, about 15%, about 10%, about 5%, about 1%, or less) or high level (e.g., greater than about 60%, about 65%, about 70%, about 75%, about 80%, about 85%, about 90%, about 95%, about 98%, about 99%, or more) of core fucosylation.

In some embodiments, a desired glycosylation pattern will include a low level (e.g., less than about 20%, about 15%, about 10%, about 5%, about 1%, or less) or high level (e.g., greater than about 60%, about 65%, about 70%, about 75%, about 80%, about 85%, about 90%, about 95%, about 98%, about 99%, or more) of a sulfated glycan.

In some embodiments, a desired glycosylation pattern will include a low level (e.g., less than about 20%, about 15%, about 10%, about 5%, about 1%, or less) or high level (e.g., greater than about 60%, about 65%, about 70%, about 75%, about 80%, about 85%, about 90%, about 95%, about 98%, about 99%, or more) of a phosphorylated glycan.

In some embodiments, a desired glycosylation pattern will include a low level (e.g., less than about 20%, about 15%, about 10%, about 5%, about 1%, or less) or high level (e.g., greater than about 60%, about 65%, about 70%, about 75%, about 80%, about 85%, about 90%, about 95%, about 98%, about 99%, or more) of a sialic acid linked to an N-acetylglucosamine.

In some embodiments, a desired glycosylation pattern will include a low level (e.g., less than about 20%, about 15%, about 10%, about 5%, about 1%, or less) or high level (e.g., greater than about 60%, about 65%, about 70%, about 75%, about 80%, about 85%, about 90%, about 95%, about 98%, about 99%, or more) of an acetylatcd glycan.

In some embodiments, it may be desirable to employ a cell line that tends to produce glycoproteins with long, unbranched oligosaccharide chains; in some embodiments, it may be desirable to employ a cell line that tends to produce glycoproteins with short, highly branched oligosaccharide chains.

Whether or not monitoring production of a particular target protein for quality control purposes, the present disclosure may be utilized, for example, to monitor cell surface glycosylation at particular stages of development, or under particular growth conditions.

In some particular embodiments of the present disclosure methods described herein can be used to characterize and/or control or compare the quality of therapeutic products. To give but one example, the present methodologies can be used to assess cell surface glycosylation in cells producing a therapeutic protein product. Particularly given that glycosylation can often affect the activity, bioavailability, or other characteristics of a therapeutic protein product, methods for assessing cellular glycosylation during production of such a therapeutic protein product are particularly desirable. Among other things, the present disclosure can facilitate real time analysis of cell surface glycosylation in production systems for therapeutic proteins.

Whether or not monitoring production of a particular target protein for quality control purposes, the present invention may be utilized, for example, to monitor glycosylation at particular stages of development, or under particular growth conditions.

In some embodiments, methods described herein can be used to characterize and/or control or compare the quality of therapeutic products. To give but one example, the present methodologies can be used to assess glycosylation in cells producing a therapeutic protein product. Particularly given that glycosylation can often affect the activity, bioavailability, or other characteristics of a therapeutic protein product, methods for assessing cellular glycosylation during production of such a therapeutic protein product are particularly desirable. Among other things, the present invention can facilitate real time analysis of glycosylation in production systems for therapeutic proteins.

Representative therapeutic glycoprotein products whose production and/or quality can be monitored in accordance with the present invention include, for example, any of a variety of hematologic agents (including, for instance, erythropoietin, blood-clotting factors, etc.), interferons, colony stimulating factors, antibodies, enzymes, and hormones, particularly those that are expressed on the cell surface.

Representative commercially available glycoprotein products include for example:

| Protein Product | Reference Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion | Amevive |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | Bexxar ® |
| antihemophilic factor | Bioclate ™ |
| human growth hormone | BioTropin ™ |
| botulinum toxin type A | Botox ® |
| Alemtuzumab | Campath ® |
| Acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune Fab, ovine | DigiFab ™ |
| Rasburicase | Elitek ® |
| Etanercept | Enbrel ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | Forteo ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | Hemofil ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ®FS |
| insulin glargine | Lantus ® |
| granulocyte macrophage colony-stimulating | Leukine ®/Leukine ® |
| factor | Liquid |
| lutropin alfa, for injection | Luveris |
| OspA lipoprotein | LYMErix ™ |
| Ranibizumab | Lucentis ® |
| Gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ |
| | (formerly LeuTech ®) |
| somatropin [rDNA] | Norditropin ®/ |
| | Norditropin |
| | Nordiflex ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| Muromomab-CD3 | Orthoclone OKT3 ® |
| human chorionic gonadotropin | Ovidrel ® |
| Peginterferon alfa-2a | Pegasys ® |
| pegylated version of interferon alfa-2b | PEG-Intron ™ |
| Abarelix (injectable suspension); gonadotropin- | Plenaxis ™ |
| releasing hormone antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell blocker | Raptiva ™ |
| combination of ribavirin and alpha interferon | Rebetron ™ |
| Interferon beta 1a | Rebif ® |
| antihemophilic factor | Recombinate ® |
| rAHF/ntihemophilic factor | ReFacto ® |
| Lepirudin | Refludan ® |
| Infliximab | Remicade ® |
| Abciximab | ReoPro ™ |
| Reteplase | Retavase ™ |
| Rituximab | Rituxan ™ |
| interferon alfa-2a | Roferon-A ® |
| Somatropin | Saizen ® |
| synthetic porcine secretin | SecreFlo ™ |
| Basiliximab | Simulect ® |
| Eculizumab | Soliris ® |
| Pegvisomant | Somavert ® |
| Palivizumab; recombinantly produced, | Synagis ™ |
| humanized mAb | |
| thyrotropin alfa | Thyrogen ® |
| Tenecteplase | TNKase ™ |
| Natalizumab | Tysabri ® |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal antibody targeting | |
| immunoglobulin-E | |
| Daclizumab | Zenapax ® |
| Ibritumomab tiuxetan | Zevalin ™ |
| Somatotropin | Zorbtive ™ |
| | (Serostim ®) |

In some embodiments, the disclosure provides methods in which cell surface glycans from different sources or samples are compared with one another. In some such examples, multiple samples from the same source are obtained over time, so that changes in glycosylation patterns (and particularly in cell surface glycosylation patterns) are monitored. In some embodiments, glycan-containing samples are removed at regular intervals. In some embodiments, glycan-containing samples are removed at about 30 second, about 1 minute, about 2 minute, about 5 minute, about 10 minute, about 30 minute, about 1 hour, about 2 hour, about 3 hour, about 4 hour, about 5 hour, about 10 hour, about 12 hour, or about 18 hour intervals, or at even longer intervals. In some embodiments, glycan-containing samples are removed at irregular intervals. In some embodiments, glycan-containing samples are removed at 5 hour intervals.

In some embodiments, one of the samples is a historical sample or a record of a historical sample. In some embodiments, one of the samples is a reference sample.

In certain embodiments, methods are provided herein which can be used to monitor the extent and/or type of cell surface glycosylation occurring in different cell cultures.

In some embodiments, cell surface glycans from different cell culture samples prepared under conditions that differ in one or more selected parameters (e.g., cell type, culture type [e.g., continuous feed vs batch feed, etc.], culture conditions [e.g., type of media, presence or concentration of particular component of particular medium(a), osmolarity, pH, temperature, timing or degree of shift in one or more components such as osmolarity, pH, temperature, etc.], culture time, isolation steps, etc.) but are otherwise identical, are compared, so that effects of the selected parameter(s) on cell surface glycosylation patterns are determined. In certain embodiments, cell surface glycans from different cell culture samples prepared under conditions that differ in a single selected parameter are compared so that effect of the single selected parameter on cell surface glycosylation patterns is determined. Among other applications, therefore, use of techniques as described herein may facilitate determination of the effects of particular parameters on cell surface glycosylation patterns in cells.

In some embodiments, glycans from different batches of a glycoprotein of interest (e.g., a therapeutic glycoprotein, particularly a cell surface glycoprotein), whether prepared by the same method or by different methods, and whether prepared simultaneously or separately, are compared. In such embodiments, the present disclosure facilitates quality control of glycoprotein preparation. Alternatively or additionally, some such embodiments facilitate monitoring of progress of a particular culture producing a glycoprotein of interest (e.g., when samples are removed from the culture at different time points and are analyzed and compared to one another). In any of these embodiments, features of the glycan analysis can be recorded, for example in a quality control record. As indicated above, in some embodiments, a comparison is with a historical record of a prior or standard batch and/or with a reference sample of glycoprotein.

In certain embodiments, the present disclosure may be utilized in studies to modify the cell surface glycosylation characteristics of a cell, for example to establish a cell line and/or culture conditions with one or more desirable cell surface glycosylation characteristics. Such a cell line and/or culture conditions can then be utilized, if desired, for production of a particular target glycoconjugate (e.g., glycoprotein) for which such glycosylation characteristic(s) is/are expected to be beneficial.

In certain embodiments, techniques of the present disclosure are applied to glycans that are present on the surface of cells. In some such embodiments, the analyzed glycans are substantially free of non-cell-surface glycans. In some such embodiments, the analyzed glycans, when present on the cell surface, are present in the context of one or more cell surface glycoconjugates (e.g., glycoproteins).

In some particular embodiments, cell surface glycans are analyzed in order to assess glycosylation of one or more target glycoproteins of interest, particularly where such target glycoproteins are not cell surface glycoproteins. Such embodiments can allow one to monitor glycosylation of a target glycoprotein without isolating the glycoprotein itself. In certain embodiments, the present disclosure provides methods of using cell-surface glycans as a readout of or proxy for glycan structures on an expressed glycoprotein of interest. In certain embodiments, such methods include, but are not limited to, post process, batch, screening or “in line” measurements of product quality. Such methods can provide for an independent measure of the glycosylation pattern of a produced glycoprotein of interest using a byproduct of the production reaction (e.g., the cells) without requiring the use of destruction of any produced glycoprotein. Furthermore, methods of the present disclosure can avoid the effort required for isolation of product and the potential selection of product glycoforms that may occur during isolation.

According to the present disclosure, techniques described herein can be used to detect desirable or undesirable glycans, for example to detect or quantify the presence of one or more contaminants in a product, or to detect or quantify the presence of one or more active or desired species.

In various embodiments the methods can be used to detect biomarkers indicative of, e.g., a disease state, prior to the appearance of symptoms and/or progression of the disease state to an untreatable or less treatable condition, by detecting one or more specific cell surface glycans whose presence or level (whether absolute or relative) may be correlated with a particular disease state (including susceptibility to a particular disease) and/or the change in the concentration of such cell surface glycans over time.

In certain embodiments, methods described herein facilitate detection of cell surface glycans that are present at very low levels in a source (e.g., a biological sample, glycan preparation, etc.). In such embodiments, it is possible to detect and/or optionally quantify the levels of cell surface glycans that are present at levels less than about 10%, 5%, 4%, 3%, 2%, 1.5%, 1%, 0.75%, 0.5%, 0.25%, 0.1%, 0.075%, 0.05%, 0.025%, or 0.01% within a population of glycans. In some embodiments, it is possible to detect and/or optionally quantify the levels of glycans comprising between 0.1% and 5%, e.g., between 0.1% and 2%, e.g., between 0.1% and 1% of a cell surface glycan preparation. In certain embodiments, it is possible to detect and/or optionally quantify the levels of cell surface glycans at between about 0.1 fmol to about 1 mmol.

In some embodiments, methods described herein allow for detection of particular linkages that are present at low levels within a population of cell surface glycans. For example, the present methods allow for detection of particular linkages that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, methods described herein allow for detection of relative levels of individual glycan species within a population of cell surface glycans. For example, the area under each peak of a liquid chromatograph can be measured and expressed as a percentage of the total. Such an analysis provides a relative percent amount of each glycan species within a population of glycans.

In some embodiments, techniques described herein may be combined with one or more other technologies for the detection, analysis, and or isolation of glycans or glycoconjugates. For example, in many embodiments, the present disclosure provides for release of glycans from cleaved glycopeptides, and subsequent direct analysis of the glycans.

### Kits

Reagents useful for the practice of one or more methods of the present disclosure may desirably be provided together, assembled in a kit. In certain embodiments, kits of the present disclosure include one or more reagents useful for liberating glycoproteins from the cell surface (e.g., one or more proteases and/or other agents) and/or supplementary components such as buffers, co-factors, etc. In certain embodiments, kits of the present disclosure include one or more reagents useful for purifying and/or further analyzing the liberated cell surface glycoprotein from the cells from which they have been liberated.

In certain embodiments, kits of the present disclosure include one or more agents useful for cleaving glycan structures from a glycopeptide (e.g., enzymes such as PNGase F, PNGase A, O-glycanase and/or Endo-H, endoglycosidases, beta-elimination, etc.). In certain embodiments, kits of the present disclosure include one or more reagents useful for purifying the cleaved glycan structures from the protein component of glycopeptides. For example, in some embodiments, kits provide one or more glycosidases.

In certain embodiments, kits of the present disclosure include one or more reagents for labeling glycan structures. For example, kits of the present disclosure may include fluorescent labels, radiolabels and/or chemiluminescent labels. In certain embodiments, kits of the present disclosure include fluorescent 2-aminobenzamide (“2-AB”).

In certain embodiments, kits of the present disclosure include one or more reagents for culturing cells (e.g., cell culture media, buffers, media components, etc.) and/or purifying cells after the cells have been cultured.

### Exemplification

### Example 1

### Use of Proteases to Release Cell Surface Glycans from Cells

The present Example demonstrates that proteases, and particularly proteolytic enzymes, can efficiently and effectively release cell surface glycans (attached to fragments of cell surface proteins cleaved by the proteases) from cells, so that they are available for analysis.

### Materials and Methods

Adherent cells or cells in suspension are isolated from cell culture media. Cell culture media may or may not contain sera. Adherent cells are removed by exposure to EDTA (1-5 mM) in phosphate buffered saline. All cells are washed at least three times with phosphate buffered saline to remove any contaminating sera proteins.

Cells are resuspended in phosphate buffered saline. Cell surface glycans are released by addition of (or resuspension into solution containing) protease. Exemplary useful proteases include trypsin, proteinase K, or combinations thereof, for example at about 0.1-2.0 mg/ml. Protease exposure may be limited (e.g., to about 15 min at 37° C.), for example to preserve cell viability.

Released glycopeptides may be separated from cells by centrifugation.

Glycans can then be released from glycopeptides, for example by denaturation and cleavage. For example, a solution of glycopeptides can be denatured by heating to 100° C. for 10 min in the presence of SDS (to 0.5%) and BME (to 50 uM) to assure complete denaturation. If proteinase K is used, then the solution can also be supplemented with protease inhibitor cocktail. Glycans are removed from glycopeptides by addition of the enzyme PNGase F and incubation at 37° C. overnight. Following PNGase F release the glycans are isolated from the peptides and enzyme by graphitized carbon chromatography. Isolated glycans are sufficiently pure to be utilized directly, or to be further derivatized.

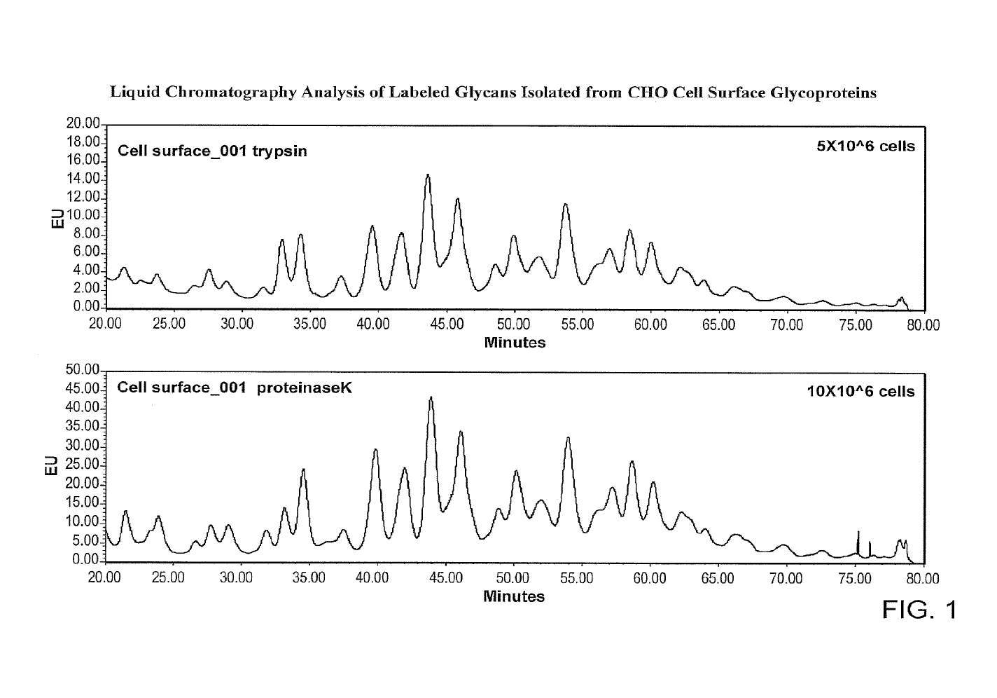

FIG. 1 shows illustrative LC spectra of cell surface glycans released from CHO cells by treatment with trypsin (Panel A) or Proteinase K (Panel B).

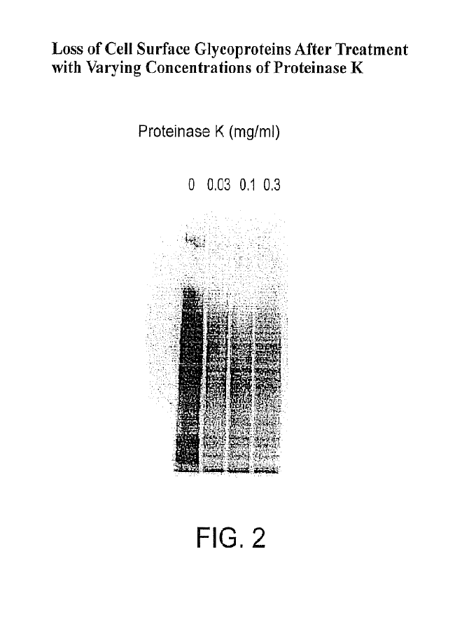

FIG. 2 shows the loss of cell-surface associated biotin from CHO cells that were treated with proteinase K.

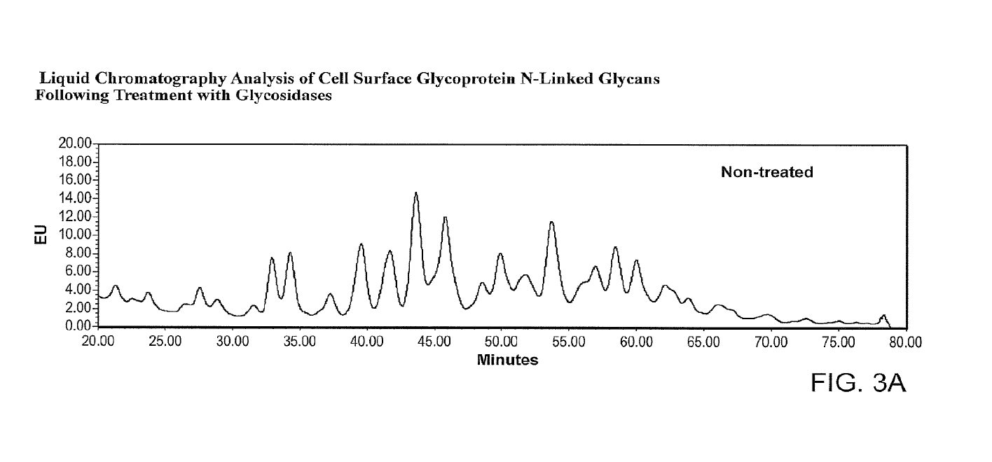

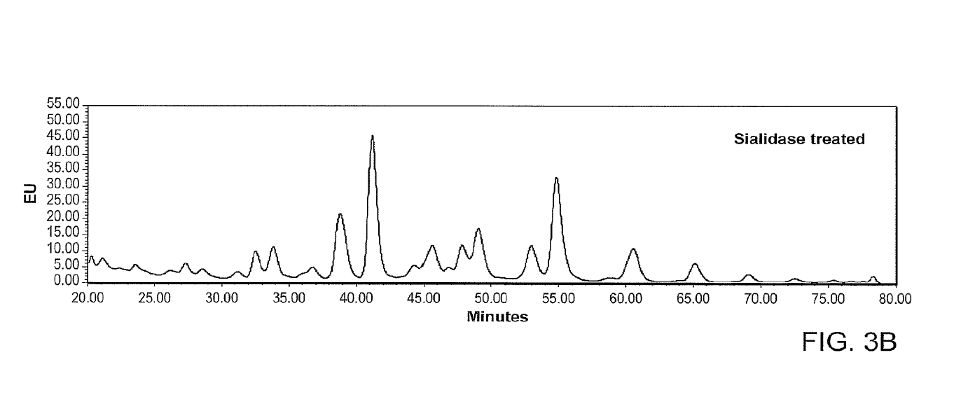

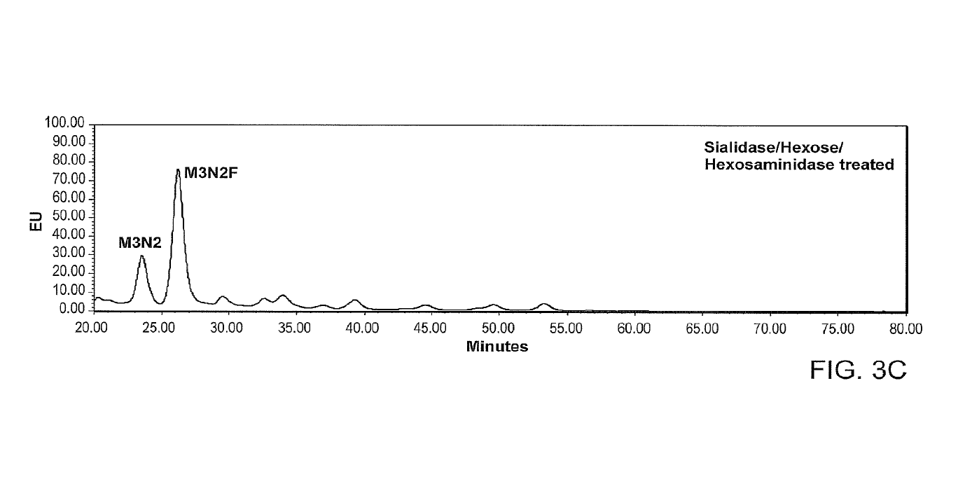

FIG. 3 shows liquid chromatography analysis of cell surface glycoprotein N-linked glycans before treatment with glycosidase (Panel A), following treatment with sialidase (Panel B), and following treatment with both sialidase and hexosaminidase (Panel C).

### Example 2

### Labeling and MS Analysis of Cell Surface Glycan Preparations

The present disclosure also provides quantification methods for determining the relative amounts of different glycan species provided in different cell-surface glycan preparations, e.g., protease digests of two different samples.

In one aspect, these methods comprise steps of: (i) reacting a first cell-surface glycan preparation prepared by a first method with a first labeling agent to provide a first labeled cell-surface glycan preparation; (ii) reacting a second cell-surface glycan preparation prepared by a second method with a second labeling agent, in which the second labeling agent has a different mass from that of the first labeling agent, to provide a second labeled cell-surface glycan preparation; (iii) combining a first amount of the first labeled cell-surface glycan preparation with a second amount of the second labeled cell-surface glycan preparation to provide a labeled cell-surface glycan mixture; (iv) analyzing the labeled cell-surface glycan mixture by mass spectrometry to provide a mass spectrum which includes pairs of peaks corresponding to glycan species labeled with the first and second labeling agents, respectively; (v) determining the relative intensities of the peaks in at least one pair of peaks; and (vi) quantifying the relative amounts of at least one glycan species provided in the first and second labeled cell-surface glycan preparations based on the relative intensities of the peaks in at least one pair of peaks.

In general it is to be understood that the mass of the first and second labeling agents may differ by any amount. Typically, the mass will differ by between 1 and 10 Daltons; however, in certain embodiments mass differences of more than 10 Daltons may be used. Small mass differences may impair the resolution of peaks from the same cell-surface glycan species. Large mass differences may cause the peaks from unrelated cell-surface glycan species to overlap. Thus, in one set of embodiments, the mass may differ by between 1 and 6 Daltons, for example between 2 and 5 Daltons. In one embodiment the mass may differ by 2 Daltons. In one embodiment the mass may differ by 3 Daltons. In one embodiment the mass may differ by 4 Daltons. In another embodiment the mass may differ by 5 Daltons.

As discussed in the present disclosure, the mass difference may be achieved by using different isotopic analogs of the same molecule. However, it is to be understood that the present disclosure is not limited to using isotopic analogs and that the same mass difference can also be achieved by using molecules with different chemical structures (e.g., two different functional tags that differ in mass by between 1 and 10 Daltons).

In certain embodiments, there is more than one labeling agent for a given step. For example, in certain embodiments, step (i) of the above method further comprises reacting the first cell-surface glycan preparation with an additional labeling agent. In certain embodiments, step (ii) of the above method further comprises reacting the second cell-surface glycan preparation with an additional labeling agent.

In certain embodiments, the second cell-surface glycan preparation is an appropriate standard. For example, the standard could be a commercially available glycan or glycan mixture (e.g., a commercially available cell-surface glycan mixture). Alternatively, the standard could be a cell-surface glycan preparation provided from a commercially available cell-surface glycoprotein therapeutic.

### Mass Spectroscopy

According to the present disclosure, once first and second labeled cell-surface glycan preparations have been prepared from different cell-surface glycoprotein preparations, they can be combined in known or unknown proportions to produce a cell-surface glycan mixture. When a quantitative comparison between two cell-surface glycan preparations is being performed then the first and second labeled cell-surface glycan preparations are combined in known proportions. When a qualitative comparison between two cell-surface glycan preparations is being performed then the first and second labeled cell-surface glycan preparations may be combined in unknown proportions. The cell-surface glycan mixture is then analyzed by mass spectroscopy.

As is well known in the art, mass spectroscopy is a method in which charged molecules are accelerated in a vacuum through a magnetic field and then sorted on the basis of mass-to-charge ratio. The ion source is the part of the mass spectrometer that ionizes the molecules under analysis. The ions are then transported by magnetic or electric fields to the mass analyzer. A variety of techniques have been developed for ionizing molecules in a mass spectrometer. Such techniques include, but are not limited to, electrospray ionization (ESI), matrix-assisted laser desorption/ionization (MALDI), fast atom bombardment (FAB), and fourier transform ion cyclotron resonance (FT). Typically, the molecules are charged by bombardment with an adjustable electron beam. Depending on the energy of the electron beam, the molecules may fragment. The process of fragmentation follows simple and predictable chemical pathways and the ions which are formed will reflect the most stable ions and radical ions which that molecule can form. Since the bulk of the ions produced in the mass spectrometer carry a single unit of charge, the value m/z is typically equivalent to the molecular weight of the molecule or fragment. The output of the mass spectrometer shows a plot of relative intensity vs the mass-to-charge ratio (m/z). The most intense peak in the spectrum is termed the base peak and all others are reported relative to its intensity.

Mass spectrometry may also be combined with one or more separation methods, such as liquid chromatography (LC) or gas chromatography (GC). In liquid chromatography mass spectrometry (LC/MS or LC-MS), compounds are separated chromatographically before they are introduced to the ion source and mass spectrometer. LC-MS differs from GC-MS in that the mobile phase is a liquid rather than a gas.

Exemplary mass spectromtetry methods include one or more tandem ionization and/or separation techniques, or combinations thereof, such as tandem mass spectrometry (MS/MS), electrospray ionization mass spectrometry (ESI-MS), electrospray ionization mass spectrometry/mass spectrometry (ESI-MS/MS), matrix-assisted laser desorption/ionization mass spectrometry (MALDI-MS), matrix-assisted laser desorption/ionization time-of-flight mass spectrometry (MALDI-TOF-MS), fast atom bombardment mass spectrometry (FAB-MS), liquid chromatography mass spectrometry (LC-MS), liquid chromatography mass spectrometry-mass spectrometry (LC-MS/MS), fourier transform ion cyclotron resonance mass spectrometry (FT-MS), and matrix-assisted laser desorption/ionization-time-of-flight/time-of-flight mass spectrometry (MALDI-TOF/TOF-MS).

Tandem mass spectrometry, or MS/MS, involves multiple steps of mass selection or analysis, usually separated by some form of fragmentation. A tandem mass spectrometer is one capable of multiple rounds of mass spectrometry. For example, one mass analyzer can isolate one molecule from many entering a mass spectrometer. A second mass analyzer then stabilizes the molecule ions while they collide with a gas, causing them to fragment by collision-induced dissociation (CID). A third mass analyzer then catalogs the fragments produced from the original molecule. Tandem MS can also be done in a single mass analyzer over time such as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD) and blackbody infrared radiative dissociation (BIRD).

In ESI, the sample solution is sprayed in a fine mist of charged droplets containing sample ions by application of a large negative or positive voltage (typically ±4.5 to ±5 kV). A flow of nitrogen drying gas is directed at droplets and individual positive or negative ions are produced. ESI accommodates a liquid flow of 1 mL/min to 1 mL/min. This ionization technique is particularly suitable for the analysis of polar, thermally labile molecules such as glycans.

MALDI is a laser-based soft ionization technique particularly suitable for the analysis of high molecular weight compounds, including glycans with relative masses up to several hundred kilodalton. For a typical MALDI analysis, the sample and matrix solutions (e.g., 2,5-dihydroxybenzoic acid (DHB), 3,5-dimethoxy-4-hydroxycinnamic acid (sinapinic acid), a-cyano-4-hydroxycinnamic acid (CHCA)), may be premixed or applied directly to the sample support (i.e., sample plate), allowed to dry and then ionized.

TOF is an analysis method used in conjunction with an ionization method which uses an electric field to accelerate the ions through the same potential, and then measures the time they take to reach the detector. If the particles all have the same charge, then their kinetic energies will be identical, and their velocities will depend only on their masses. Lighter ions reach the detector first.

FAB refers to a “soft” ionization technique used in mass spectrometry in which a molecule and a non-volatile chemical protection environment (liquid matrix such as glycerol or 3-nitrobenzyl alcohol) mixture are bombarded by a ˜8 KeV particle beam of usually inert gas such as argon or xenon. Polar molecules, such as glycans may be analyzed by this method.

FT is another analysis method used in conjunction with an ionization method wherein ions, passing through a series of pumping stages at increasingly high vacuum, travel through magnetic field, and subsequently are bent into a circular motion in a plane perpendicular to the field by the Lorentz Force. The frequency of rotation of the ions is dependent on their m/z ratio. Excitation of each individual m/z is achieved by a swept RF pulse across the excitation plates of the cell. When the RF goes off resonance for that particular m/z value, the ions drop back down to their natural orbit (relax), resulting in an FID signal. Deconvolution of this signal by FT methods results in the deconvoluted frequency vs. intensity spectrum which is then converted to the mass vs. intensity spectrum (the mass spectrum). Due to the ion-trap nature of FT-MS, it is possible to measure the ions without destroying them, this enables further experiments to performed on the ions.

In certain embodiments, the labeled cell-surface glycan mixture is analyzed by a method selected from the group consisting of electrospray ionization mass spectrometry (ESI-MS), electrospray ionization mass spectrometry/mass spectrometry (ESI-MS/MS), matrix-assisted laser desorption/ionization mass spectrometry (MALDI-MS), matrix-assisted laser desorption/ionization time-of-flight mass spectrometry (MALDI-TOF-MS), fast atom bombardment mass spectrometry (FAB-MS), tandem mass spectrometry (MS/MS), liquid chromatography mass spectrometry (LC-MS), liquid chromatography mass spectrometry-mass spectrometry (LC-MS/MS), fourier transform ion cyclotron resonance mass spectrometry (FT-MS), and matrix-assisted laser desorption/ionization-time-of-flight/time-of-flight mass spectrometry (MALDI-TOF/TOF-MS).

In certain embodiments, the labeled cell-surface glycan mixture is analyzed by a method selected from the group consisting of electrospray ionization mass spectrometry (ESI-MS), electrospray ionization mass spectrometry/mass spectrometry (ESI-MS/MS), matrix-assisted laser desorption/ionization mass spectrometry (MALDI-MS), matrix-assisted laser desorption/ionization time-of-flight mass spectrometry (MALDI-TOF-MS), liquid chromatography mass spectrometry (LC-MS), liquid chromatography mass spectrometry-mass spectrometry (LC-MS/MS), and matrix-assisted laser desorption/ionization-time-of-flight/time-of-flight mass spectrometry (MALDI-TOF/TOF-MS). However, in certain embodiments, LC-MS and LC-MS/MS as a mass spectrometric method are excluded.

A mass spectrum of the cell-surface glycan mixture will include pairs of peaks that correspond to cell-surface glycan species labeled with the first and second labeling agents, respectively. These peaks will be separated by the mass difference of the first and second labeling agents that were used in preparing the first and second labeled cell-surface glycan preparations. For example, if the mass difference of the first and second labeling agents was 4 Daltons (e.g., the first labeling agent was a d₄ analog of the second labeling agent) then the peaks will be separated by 4/z in the mass spectrum.

The relative amounts of a given cell-surface glycan species that were provided by the first and second labeled cell-surface glycan preparations can be readily determined based on the relative peak intensities. In one embodiment this can involve comparing peak heights. In another embodiment this can involve comparing peak areas. This might require deconvoluting overlapping peaks, e.g., when the mass difference between the first and second labeling agents is smaller than the peak width.

### Equivalents and Scope

Those skilled in the art will recognize, or be able to ascertain using no more than routine experimentation, many equivalents to the specific embodiments of the disclosure, described herein. The scope of the present disclosure is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the disclosure, or aspects of the disclosure, is/are referred to as comprising particular elements, features, etc., certain embodiments of the disclosure or aspects of the disclosure consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any exoglycosidase, any glycosidic linkage, any reaction condition, any method of purification, any method of product analysis, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. A method comprising:
exposing a cell that has at least one cell surface glycan attached to a cell surface glycoprotein to a protease such that the cell surface glycoprotein is cleaved and a glycopeptide comprising the cell surface glycan is liberated, wherein exposure to the protease does not substantially rupture the cell membrane; and isolating or analyzing the cell surface glycan.

2. The method of claim 1, wherein the cell is exposed to a labeling agent, such that the cell surface glycan is labeled, prior to the step of exposing, wherein the glycopeptide that is liberated comprises the labeled cell surface glycan.

3. The method of claim 2, wherein the step of isolating or analyzing comprises:
liberating the cell surface glycan from the liberated cell surface glycoprotein to generate a liberated cell surface glycan and a liberated peptide backbone; and
isolating or analyzing at least one moiety selected from the group consisting of the liberated cell surface glycan and the liberated peptide.

4. (canceled)

5. The method of claim 2, wherein at least about 75% of cell membranes remain intact, as determined by trypan blue exclusion.

6.-9. (canceled)

10. The method of claim 2, wherein the protease comprises one or more proteolytic enzymes selected from the group consisting of: proteinase K, trypsin, and combinations thereof.

11.-13. (canceled)

14. The method of claim 3, wherein the step of liberating the cell surface glycan comprises subjecting the liberated cell surface glycopeptide to an agent selected from the group consisting of: PNGase F, PNGase A, Endo-H, O-glycanase, hydrazine, endoglycosidases, beta-elimination, and combinations thereof.

15. The method of claim 14, wherein the liberated cell surface glycopeptide is denatured prior to the step of isolating the liberated cell surface glycan.

16. The method of claim 14, wherein the step of isolating the liberated cell surface glycan comprises a step of:
subjecting the liberated cell surface glycan to graphitized carbon chromatography.

17. The method of claim 3, wherein the step of isolating or analyzing the liberated cell surface glycan comprises performing a characterization method selected from the group consisting of: NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof.

18. The method of claim 2, wherein the cell surface glycan comprises a moiety selected from the group consisting of an N-linked glycan; an O-linked glycan; a complex glycan; a glycan that comprises at least one sialic acid residue; a high-mannose glycan; and a hybrid glycan.

19.-23. (canceled)

24. A method comprising:
providing a first cell that has at least one first cell surface glycan attached to a first cell surface glycoprotein;
providing a second cell that has at least one second cell surface glycan attached to a second cell surface glycoprotein;
exposing the first and second cells to a protease such that
(i) the first and second cell surface glycoproteins are cleaved,
(ii) a first glycopeptide comprising the first cell surface glycan is liberated, and
(iii) a second glycopeptide comprising the second cell surface glycan is liberated, wherein exposure to the protease does not substantially rupture the cell membranes of the first and second cells;
liberating the first and second cell surface glycans from the liberated first and second cell surface glycopeptides;
analyzing the liberated first and second cell surface glycans; and
comparing the liberated first and second cell surface glycans.

25.-26. (canceled)

27. The method of claim 24, wherein at least one of the following is satisfied;
(i) the first cell is exposed to a labeling agent, such that the first cell surface glycan is labeled, and
(ii) the second cell is exposed to a labeling agent, such that the second cell surface glycan is labeled.

28. The method of claim 24 further comprising steps of:
isolating a first peptide backbone resulting from liberating the first cell surface glycan from the first cell surface glycopeptide;
isolating a second peptide backbone resulting from liberating the second cell surface glycan from the second cell surface glycopeptide;
analyzing the isolated first and second peptide backbones; and
comparing the isolated first and second peptide backbones.

29. The method of claim 24 wherein the first cell and second cells are grown under different cell culture conditions, wherein the cell culture conditions differ in at least one parameter selected from the group consisting of: cell type, culture type, culture time, media type, osmolarity, pH, temperature, isolation steps, and combinations thereof.

30. The method of claim 24 wherein the first cell comprises a first sample from a cell culture, taken at a first time point and the second cell comprises a second sample from the cell culture, taken at a second time point.

31. The method of claim 24 wherein the first cell comprises a sample from a cell culture and the first cell comprises a reference standard.

32. (canceled)

33. The method of claim 24, wherein (i) the first cell comprises a plurality of first cells, (ii) the second cell comprises a plurality of second cells, and (iii) wherein at least about 75% of first cell membranes, at least about 75% of second cell membranes, or both, remain intact, as determined by trypan blue exclusion.

34.-37. (canceled)

38. The method of claim 24, wherein the protease comprises one or more proteolytic enzymes selected from the group consisting of: proteinase K, trypsin, and combinations thereof.

39.-41. (canceled)

42. The method of claim 24, wherein the step of liberating the first or second cell surface glycan comprises subjecting the first or second liberated cell surface glycoprotein to an agent selected from the group consisting of: PNGase F, PNGase A, Endo-H, O-glycanase, hydrazine, endoglycosidases, beta-elimination, and combinations thereof.

43.-44. (canceled)

45. The method of claim 3, wherein the step of analyzing comprises a characterization method selected from the group consisting of: NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof.

46. The method of claim 24, wherein the first or second cell surface glycan comprises a moiety selected from the group consisting of an N-linked glycan; an O-linked glycan; a complex glycan; a glycan that comprises at least one sialic acid residue; a high-mannose glycan; and a hybrid glycan.

47.-51. (canceled)

52. The method of claim 24, wherein the step of analyzing comprises a characterization method selected from the group consisting of: NMR, mass spectrometry, liquid chromatography, 2-dimensional chromatography, SDS-PAGE, antibody staining, lectin staining, monosaccharide quantitation, capillary electrophoresis, fluorophore-assisted carbohydrate electrophoresis (FACE), micellar electrokinetic chromatography (MEKC), exoglycosidase or endoglycosidase treatments, and combinations thereof.
